(19) United States
(12) Patent Application Publication
(10) Pub. No.: US 2021/0299206 A1
(43) Pub. Date: Sep. 30, 2021

(54) VAGINAL CARE COMPOSITIONS

(71) Applicant: The Procter & Gamble Company, Cincinnati, OH (US)

(72) Inventors: Travis Kyle HODGDON, Cincinnati, OH (US); Leo Timothy LAUGHLIN, II, Mason, OH (US); Samantha Chen-Yee WANG, Cincinnati, OH (US)

(21) Appl. No.: 17/116,132

(22) Filed: Dec. 9, 2020

Related U.S. Application Data
(63) Continuation of application No. PCT/US2019/039440, filed on Jun. 27, 2019.
(60) Provisional application No. 62/691,805, filed on Jun. 29, 2018.

(51) Int. Cl. A61K 36/899; A61K 36/49; A61K 36/61; A61K 36/42; A61K 36/63; A61K 36/738; A61K 9/107

(52) U.S. Cl. CPC A61K 36/899; A61K 36/49; A61K 36/61; A61K 9/107; A61K 36/63; A61K 36/738; A61K 36/42

(57) ABSTRACT

A natural vaginal care composition is provided. The natural composition includes a biobased vaginal care agent; a biobased thickener; and at least 60% water. The natural composition may optionally include a biobased preservative, a biobased emulsifier, and a biobased pH adjuster. The natural vaginal care composition is suitable for treating symptoms of vaginal atrophy and/or dryness.

### TECHNICAL FIELD

Vaginal care compositions made from all naturally derived ingredients are provided.

### BACKGROUND

Estimates indicate that by 2030 there will be about 1.2 billion menopausal and post-menopausal women in the world. Given that the average age at which menopause occurs has remained the same and life expectancy among women has generally increased, the number of post-menopausal women is expected to grow. As such, it is expected that there will be a growing need for treating the conditions and symptoms experienced by peri-menopausal, menopausal, post-menopausal women.

There are a variety of vaginal care products on the market for treating conditions of vaginal health such as vaginal atrophy and vaginal dryness. Many of these conventional compositions are applied in the vaginal canal or on or around sensitive vaginal tissue. However, at least some vaginal care compositions contain synthetic ingredients derived from silicone or petroleum. A recent social movement to use only natural personal care products has led to a reluctance by some consumers to place synthetic materials on or in their body, especially sensitive or intimate areas of the body. Thus, there is a need for a vaginal care product made only from biobased ingredients (i.e., ingredients that are derived from plants or animals)

Accordingly, it would be desirable to provide a vaginal care composition that includes mostly or only natural and/or biobased ingredients. It would also be desirable to provide a vaginal care composition that includes a natural and/or biobased vaginal care agent for treating a symptom of vaginal atrophy. It would further be desirable to provide vaginal care composition that includes one or more natural and/or biobased ingredients for treating vaginal dryness.

### SUMMARY

A natural vaginal care composition is provided. The natural vaginal care composition contains a biobased vaginal care agent, a biobased thickener, and at least 60% water. In some instances, the natural vaginal care composition is in the form of an oil-in-water emulsion and includes a biobased acid or salt thereof, a biobased emulsifier, and/or a biobased preservative. The natural vaginal care compositions disclosed herein can be used to treat symptoms of vaginal atrophy and/or vaginal dryness.

### DETAILED DESCRIPTION

Conventional vaginal care compositions typically contain a variety of synthetic ingredients (silicone oils, silicone elastomers, petroleum-based polymers and fatty acids, synthetic vitamins, etc.), which may be undesirable for use by some consumers. And while natural, anhydrous vaginal care compositions may be known, they can be difficult to apply and can leave an undesirable heavy, coated feeling after application. Creating an oil-in-water emulsion using predominantly or exclusively natural and/or bioderived ingredients would lead to a vaginal care formulation that appeals to consumers who are sensitive to sustainable products while providing a use experience that is superior to traditional anhydrous balms. However, oil-in-water can present processing and storage challenges. For example, oil-in-water emulsion systems generally require the use of emulsifiers and/or preservatives to stabilize the emulsion and prevent the growth of undesirable microorganisms. To address this need, the natural vaginal care compositions herein are formulated to contain a biobased vaginal care agent, a biobased thickener, at least 60% water, and, in some instances, a biobased emulsifier and/or biobased preservative, as well as other optional ingredients that are suitable for use in a vaginal care composition.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

All percentages disclosed herein are by weight of the vaginal care composition, unless specifically stated otherwise. All ratios are weight ratios, unless specifically stated otherwise. All ranges are inclusive and combinable. The number of significant digits conveys neither a limitation on the indicated amounts nor on the accuracy of the measurements. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive of narrower ranges; delineated upper and lower range limits are interchangeable to create further ranges not explicitly delineated.

The vaginal care compositions herein can comprise, consist essentially of, or consist of, the components as well as optional ingredients described herein. As used herein, “consisting essentially of means that the composition, method, or components thereof may include additional ingredients or features, but only if the additional ingredients or features do not materially alter the basic and novel characteristics of the claimed compositions or methods. As used in the description and the appended claims, the singular forms “a,” “an,” and “the” are intended to include the plural forms as well, unless the context clearly indicates otherwise.

“About” when used in the context of a parameter or range means a value that is within 30% of the stated value (e.g., with 25%, 20%, 15%, 10%, 5%, 2% or even within 1%).

“Biobased” refers to a product or material that is composed in whole or in significant part of biological products and/or renewable materials obtained from plants and/or animals and derivatives thereof, and excludes silicone-based materials and petroleum-based materials. It is to be appreciated that the biobased materials herein include materials derived directly from a plant or animal source and then further processed (e.g., extraction, chemical synthesis, polymerization).

“Derivative” means a molecule similar to that of another one, but differing from it with respect to a certain functional moiety (e.g., esters, ethers, amides, amines, carboxylic acids, hydroxyls, acetyls, thiols, halogens, and/or salts of the relevant molecule).

“Dermatologically acceptable” means that the compositions or components thereof so described are suitable for use in contact with human tissue without undue toxicity, incompatibility, instability, allergic response, and the like.

“Effective amount” means an amount of a compound or composition sufficient to significantly induce a positive or desired benefit, (e.g., a positive skin or feel benefit, reverse the expression of a gene, group of genes and/or gene expression signature), including independently or in combinations the benefits disclosed herein, but low enough to avoid serious side effects, i.e., to provide a reasonable benefit to risk ratio, within the scope of sound judgment of the skilled artisan.

“Introitus” refers to the vaginal opening and up to 35 millimeters (mm) into the vaginal canal from the vaginal opening.

“Labia” refers, generally, to the labia majora and the labia minora.

“Menopause” refers to the biological condition where a woman does not experience a menstrual period for 12 consecutive months and no other biological or physiological cause can be identified. Post-menopausal women typically have a blood level of estradiol of less than 30 pg/ml.

“Mucoadhesion” refers to the phenomenon where a natural or synthetic substance applied to a mucosal epithelium adheres to the mucus layer, typically creating a new interface. Generally, mucoadhesion can be achieved via physical or chemical processes or both, for example, as described in J. Controlled Release, Vol. 2 (1982) pp. 257 and J. Controlled Release, Vol. 18 (1992) pp. 249.

“Natural” refers to a material that is found in nature and is minimally altered prior to being incorporated into a finished product. Examples of natural materials include honey, deionized water, and beeswax.

“Natural products” or “natural compositions” herein are products or compositions that contains at least 50% natural materials and less than 5% silicone and/or petroleum-based materials.

“Non-volatile” means that the material exhibits a vapor pressure of less than 0.2 mm Hg at 25° C. and one atmosphere and/or a material that has a boiling point at one atmosphere of at least 300° C.

“Petroleum-based” refers to a material obtained from a fossil fuel such as petroleum, natural gas, and coal. Petroleum-based products are typically obtained by physically and/or chemically modifying the fossil fuel (e.g., refining, polymerizing, or cracking).

“Progesterone agent” means any natural or synthetic progesterone hormone, metabolites thereof, analogues thereof, progesterone precursors and/or any compound which binds to a progesterone receptor or which otherwise exhibits at least mild or weak progesterone-like effects, including selective progesterone receptor modulators (“SPRM”) such as, for example, telapristone.

“Silicone-based” refers to a material that contains silicone.

“Substantially free” means a component or material is present in amount less than 0.5% (e.g., 0.1%, 0.05%, 0.025%, 0.01%, or even less than 0.001%) by weight of the vaginal care composition.

“Vaginal care agent” means any substance, as well any component or derivative thereof, that is useful for treating or ameliorating one or more symptoms of vaginal atrophy or dryness and is suitable for application to the introitus, labia, vulva, vestibule, and external urogenital tract.

“Vaginal care composition” means any composition comprising a vaginal care agent.

“Vaginal tissue” means epithelial tissue associated with one or more of the introitus, vulva, vestibule, labia, and external urogenital tract.

“Volatile” means that the material exhibits a vapor pressure of 0.2 mm of mercury or more at 25° C. and one atmosphere.

### Natural Vaginal Care Composition

The vaginal care compositions described herein are natural products that include at least 50%, by weight, natural ingredients and less than 5% silicone- and/or petroleum-based materials (e.g., less than 4%, 3%, 2%, 1%, 0.5%, or even 0). In some instances, excluding water, the natural vaginal care composition includes at least 50% (e.g., 50%-100%, 60%-90%, or even 70% to 80%), by weight of the non-aqueous portion of the composition, biobased ingredients. In some instances, the natural vaginal care composition includes 0.01% to 40% (e.g., 0.1% to 30%, 1% to 20% or even 5% to 15%) of a biobased vaginal care agent (e.g., a biobased chronic active and/or a biobased emollient), a biobased thickener, an optional biobased emulsifier, and at least 60% water. When the natural composition herein is in the form of an emulsion, as described in more detail below, it may be desirable to use a biobased emulsifier. The amount of biobased material present in the natural vaginal care composition may be determined according to ASTM D6866 (“Assessment of the Bio-based Content of Materials”), which quantitatively determines the amount of biobased carbon (i.e., non-petroleum derived) relative to the total carbon content in a material or product via a carbon dating technique. Thus, the biobased materials and compositions described herein generally have a percent biobased carbon content of 70% to 100% (e.g., 80% to 95% or about 90%).

The natural vaginal care compositions described herein are intended for topical application to the introitus and/or the labia. The present compositions provide a suitable dry feel, moisturization/emolliency, lubricity, and/or vaginal skin health benefit. A biobased vaginal care agent may be combined with a biobased and/or natural dermatologically acceptable carrier, along with any optional ingredients (e.g., preservatives, rheology modifiers, emulsifiers, humectants, lubricants, moisturizers, feel modifiers, pH agents, emollients, vitamins), using conventional methods of making such compositions. In some instances, the natural vaginal care composition may be provided in the form of a spreadable gel, serum, lotion, paste or cream.

The vaginal care agent(s) included in the compositions herein are intended to provide a vaginal care benefit. In some instances, vaginal care actives may be selected to inhibit, stop, or even reverse symptoms associated with vaginal atrophy and/or vaginal dryness. For example, co-pending U.S. Provisional App. Nos. 62/676,054 and 62/676,068, disclose that the labia majora and the vaginal introitus undergo markedly different changes in gene expression during menopause. Thus, it may be desirable to specifically select a biobased vaginal care agent for treating the unique changes in gene expression associated with the introitus, the labia, or both. When botanical (i.e., plant derived) ingredients are included in the present compositions, they may be provided as extracts obtained from any suitable part of the plant (e.g., leaf, root, stem, flower, seeds) using methods known to those skilled in the art of making botanical extracts. Additionally or alternatively, fresh and/or dried plant material and/or plant serum fraction(s) may be used. Some nonlimiting examples of methods for producing plant extracts and plant serum fractions are disclosed, respectively, in U.S. Pat. Nos. 9,358,263 and 7,442,391. When activated microorganisms (e.g., M. luteus) are included in the compositions herein, they may be activated by heat or UV radiation, for example, as described in U.S. Pat. No. 9,913,800.

The vaginal care compositions herein are generally free or substantially free of ingredients that may be irritating to vaginal tissues, such as certain retinoids, ethanol, sunscreen agents, perfumes, and particulates having an average particle size of more than 125 microns. The vaginal care compositions herein are also free or substantially free of estrogen and/or progesterone agents, due to the undesirable side effects that have been reported for these ingredients.

The natural vaginal care composition may be in the form of a solution, dispersion, or emulsion. It may be particularly desirable to provide the composition in the form of an oil-in-water emulsion to provide a sensorial feel that is light and non-greasy, but still delivers moisturization and lubricity without the undesirable feel properties commonly associated with some conventional vaginal care compositions (e.g., stickiness or heavy residue feeling). Suitable oil-in-water emulsions herein may comprise a continuous aqueous phase of more than 60% by weight of the composition, and the remainder being the dispersed oil phase. The aqueous phase may include 1% to 99% water, based on the weight of the aqueous phase, along with any water soluble and/or water miscible ingredients. The dispersed oil phase is typically present at less than 30% by weight of composition (e.g., 1% to 20%, 2% to 15%, 3% to 12%, 4% to 10%, or even 5% to 8%) to help avoid some of the undesirable feel effects of oily compositions. The oil phase of the vaginal care compositions herein may include one or more biobased chronic actives and/or emollients.

### Biobased Chronic Actives

The vaginal care agent present in the vaginal care compositions herein may include 0.0001%-15% (e.g., 0.005%-12%, 0.1%-10%, 0.5%-8%, or even 1%-5%) by weight of one or more biobased vaginal care actives for treating a chronic vaginal health condition such as, for example, a symptom of vaginal atrophy and/or vaginal dryness. Some nonlimiting examples of biobased chronic actives that may be suitable for treating symptoms of a vaginal condition such as vaginal atrophy and/or vaginal dryness include: sweet chestnut (Castanea sativa); cabrueva (Myrocarpus frondosus and/or Myrocarpus fastigiatus); mastic tree gum (Pistacia lentiscus); Chinese cinnamon (Cinnamomum cassia); bamboo; peony (Paeonia suffruticosa); hydrolyzed soybean fiber; yeast extract; red algae (Gelidium cartilagineum); evening primrose (Oenothera biennis); Chinese cabbage (Brassica rapa); Greek horehound (Ballota pseudodictamus); lamb's ear (Stachys byzantina); Alpinia speciose; hops (Humulus lupulus); plankton extract (e.g.; phormistin G); elder flower (Sambucus nigra); jiaogulan (Gynostemma pentaphyllum); Cnidium officinale; plantain (Plantago lanceolate); flax (Linum usitatissimum); yerba mansa (Anemopsis californica); mixture of tea berry and orange flower; mixture of white nectarine and Coral Flower; peach (Prunus persica); senna (Senna alexandrina); tangerine (Citrus tangerina); bamboo cactus; grass-leaf sweet flag (Acorus gramineus); Coriander (Coriandrum sativum); oregano (Origanum vulgare); European goldenrod (Solidago virga); conduragno (Marsdenia cundurango); Australian sandalwood (Santalum spicatum); lemon-scented tea tree (Leptospermum petersonii); Oregon grape (Mahonia aquifolium); parsley (Petroselinum crispum); Asian ginseng (Panax notoginseng and/or Panax ginseng); cardamom (Amomum subulatum and/or Amomum tsao-ko); honeyed pear silk; common reed (Phragmites australis); Atractylodes lancea; lavender (Lavandula angustifolia); nettle; marigold (Calendula officinalis); comfrey (Symphytum officinale); blueberry; yellow sweet clover (Melilotus officinalis); linden tree; pear; apple; nutmeg (Myristica fragrans); lyceum; kiwi (Actinidia deliciosa); St. Ignatius tree (Strychnos ignatia); eucalyptus; Cynanchum pycnostelma; cucumber (Cucumis sativus); aloe (Aloe perfoliate); peppermint (Mentha piperita); basil (Ocimum basilicum); olive (Olea europaea); perilla; false unicorn (Chamaelirium luteum); champagne yeast (e.g., Eternixine® from Ashland, Inc); rue (Ruta graveolens); plum; mixture of grapefruit, bergamot, and verbena; mandarin balm; sugar cane; terpineol (e.g., Melafresh® brand terpineol from LucasMeyer Cosmetics); rose hip; Fo ti (Fallopia multiflora); Black pepper (Piper nigrumi); green coffee bean; kudzu; frankincense oil; onion; guarana (Paullinia cupana); mango (Mangifera indica); chamomile; bilberry (Vaccinium myrtillus); skullcap root; mandarin orange (Citrus reticulata or Citrus nobilis), activated Micrococcus luteus, and derivatives and combinations of these.

### Biobased Emollients

The vaginal care agent present in the natural vaginal compositions herein may include 0.1% to 30% (e.g., 0.5% to 25%, 1% to 20%, 5% to 15% or even about 10%) by weight of the vaginal care composition of a biobased emollient to provide a moisturization benefit to vaginal tissue and a lubricious feel during application. The biobased emollient may be a botanical oil (i.e., an oil derived from plant material) or an animal-based fat or oil. Biobased fatty acids can be obtained directly from a plant or animal source (e.g., plant seed or fruit), or they may be formed from a biobased starting material. Fatty acids in the biobased emollient may be saturated, unsaturated, polyunsaturated, or hydrogenated. The biobased emollient may include volatile oils (e.g., essential oils) and/or non-volatile oils. In some instances, it may be particularly desirable to include a botanical oil that contains omega-3 (e.g., α-linolenic acid) and/or omega-6 fatty acids. For example, Camelina sativa seed oil, evening primrose oil, sunflower seed oil, and safflower oil are generally considered to be rich in omega-3 and/or omega-6 fatty acids.

In some instances, it may be desirable for the emollient to have an oil stability index (“OSI”) of at least 10 hours (e.g., at least 14 hours or 18 hours) at 110° C. A common measure for monitoring oxidative stability is the development of hydroperoxides (peroxide value or PV) over time. Oxidative stability can also be expressed in terms of the time required to obtain secondary oxidation products when aerating a sample at elevated temperature. This time, called the Oil Stability Index or OSI, is normally measured at 110 C [Amer Oil Chem Soc Oil Stability Index Method Cd 12b-92] using the Rancimat instrument (Brinkmann Instruments, Inc.), the OSI instrument (Omnion; Inc.); or equivalent. Some non-limiting examples of emollients with an OSI of at least about 10 hours that may be suitable for use herein include oleic canola oil (Brassica campestris; B. napus; B. rapa) characterized by having an oleic content greater than 70; manila kernel oil (Sclerocarya birrea); palm oil (Elaeis Guineensis Oil); palm olein; palm stearin; palm superolein; pecan oil; pumpkin seed oil; oleic safflower oil (Carthamus tinctorius) characterized by having an oleic content of greater than about 30% and omega-6 fatty acid content of less than about 50%; sesame oil (Sesamum indicum; S. oreintale); oleic soybean oil; oleic sunflower oil (Helianthus annus) characterized by having an oleic content of greater than about 40%; and mixtures thereof.

Some non-limiting examples of biobased emollients that may be suitable for use in the present compositions include anise oil, balm mint oil, bee balm oil, birch oil, bitter almond oil, bitter orange oil, calendula oil, nutmeg oil, caraway oil, chamomile oil, cinnamon oil, clove leaf oil, clove oil, coriander oil, Cypress oil, eucalyptus oil, fennel oil, gardenia oil, geranium oil, ginger oil, grapefruit oil, hyptis oil, juniper oil, kiwi oil, laurel oil, lavender oil, lemongrass oil, lemon oil, lovage oil, mandarin orange oil, musk rose oil, nutmeg oil, olibanurn, orange flower oil, orange oil, peppermint oil, pine oil, rose hips oil, rosemary oil, rose oil, rue oil, sage oil, sandalwood oil, sassafras oil, spearmint oil, sweet marjoram oil, sweet violet oil, tea tree oil, thyme oil, wild mint oil, yarrow oil, ylang ylang oil, apricot kernel oil, avocado oil, babassu oil, borage seed oil, butter, camellia oil, canola oil, triglycerides (e.g., caprylic/capric/lauric triglyceride, caprylic/capric/linoleic triglyceride, caprylic/capric/stearic triglyceride, caprylic/capric triglyceride, lauric/palmitic/oleic triglyceride, and oleic/linoleic triglyceride, oleic/paimitic/lauric/myristic/linoleic triglyceride), carrot oil, cashew nut oil, castor oil, cherry pit oil, cocoa butter, coconut oil, fish oil (e.g., cod liver oil and orange roughy oil), corn oil, wheat germ oil, cottonseed oil, evening primrose oil, glyceryl triacetyl hydroxystearate, glyceryl triacetyl ricinoleate, glycosphingolipids, grape seed oil, hazelnut oil, safflower oil, sunflower seed oil, coconut oil, cottonseed oil, lard, menhaden oil, mink oil, palm oil, peanut oil, soybean oil, tallow, vegetable oil, lard, lanolin and lanolin derivatives, lesquerella oil, macadamia nut oil, maleated soybean oil, meadowfoarn seed oil, menhaden oil, mink oil, moringa oil, mortierella oil, oleostearine, olive oil, ornental lipids, palm kernel oil, palm oil, peach kernel oil, peanut oil, pentadesma butter, pistachio nut oil, rapeseed oil, rice bran oil, sesame oil, shea butter, soybean oil, sphingolipids, sweet almond oil, tall oil, tallow, tribehenin, tricaprin, tricaprylin, triheptanoin, arachidic acid, behenic acid, capric acid, caproic acid, caprylic acid, menhaden acid, hydroxystearic acid, isostearic acid, lauric acid, linoleic acid, linolenic acid, myristic acid, oleic acid, palmitic acid, palm kernel acid, pelargonic acid, ricinoleic acid, stearic acid, undecanoic acid, undecylenic acid, derivatives (e.g., hydrogenated or acid forms) and mixtures thereof.

Some non-limiting examples of commercially-available biobased oils which may be suitable for use as emollients in the compositions herein include Preference® brand partially hydrogenated soybean oil; NuSun® brand mid-oleic sunflower oil, Clear Valley® brand high oleic sunflower oil, Clear Valley® 65 brand high oleic canola oil, and Clear Valley® 75 brand high oleic canola oil, all from Cargill; Oleocal C104 brand high oleic canola oil from Lambert Technology; marula kernel oil from Arch Personal Care; Plenish® brand high oleic soybean oil from Pioneer; low linolenic soybean oil from Asoyia; and refined sesame oil from Dipasa.

### Biobased Thickener

In some instances, the natural vaginal care compositions herein include a biobased thickener to facilitate achieving the desired viscosity and/or other rheological properties in combination with the other ingredients in the vaginal care composition. The vaginal care compositions herein may have a viscosity suitable for dispensing onto an applicator or fingers without dripping or runniness and is conducive to spreading onto the vaginal tissues of interest using hand(s), finger(s) and/or an applicator without undue effort. For example, the vaginal care composition may have a viscosity of 2,000 cps to 200,000 cps; in some examples, 5,000 cps to 150,000 cps; and in some examples, 20,000 cps to 90,000 cps, or any range formed by any of the preceding values. The biobased thickener may be present in the vaginal care composition at 0.1% to 10% (e.g., 0.5% to 8%, 1% to 5%, or even 2% to 4%) by weight of the vaginal care composition.

Some non-limiting examples of rheology modifiers that are suitable for use herein include natural and biobased waxes (e.g., beeswax), starches, gums, and/or celluloses. Biobased starches are typically sourced from plants such as corn, wheat, potato, rice, cassava, and tapioca, but other sources of biobased starch may be suitable as well. Biobased starches may be unmodified, modified, or partially degraded. Some non-limiting examples of modified starch include cationic starch, hydroxyehtyl starch, carboxymethylated starch, and polylactic acid graft-starch and polycaprylactone graft starch. Some nonlimiting examples of degraded starches include dextrin and maltodextrin (e.g., with a dextrose equivalent of 30 or lower).

Biobased gums are typically obtained from natural sources such as trees, plants, animals and seeds, and may be modified or fermented. Some non-limiting examples of natural gums include gum acacia, gum tragacanth, gum karaya, gum ghatti, pectin, carrageenan (e.g., iota or lambda), agar, furcellaran, konjac gum, gelatin, guar gum, locust bean gum, tara gum, cassia gum, mesquite gum, tamarind seed gum, quince seed gum, flaxseed gum, phyllium seed gum and oat gum. Some non-limiting examples of fermented gums include xanthan gum, dextran and pullulan.

Some nonlimiting examples of cellulosic rheology modifiers include nanocrylstalline cellulose, microfibrillated cellulose, alkyl cellulose, alkyl alkoxyalkyl cellulose, carboxyalkyl cellulose, alkyl carboxyalkyl cellulose. Particularly suitable example of cellulosic polymer(s) include carboxymethyl cellulose, methyl cellulose, methyl hydroxyethyl cellulose, methyl carboxymethyl cellulose, and mixtures thereof.

### Biobased Emulsifier

When the natural vaginal care composition is an emulsion, a biobased emulsifier may be included to help provide a stable emulsion. For example, the emulsion can include 0.01% to 10% or even 0.1% to 5%, of a nonionic and/or anionic emulsifier. Biobased emulsifiers suitable for use herein can include a wide variety of materials having an HLB value in the range of from about 8 to about 14. Some non-limiting examples of biobased emulsifier groups include alkyl ether sulfates, fatty alcohol ethoxylates, alkyl polyglycosides, and mixtures thereof.

In some instances, the natural vaginal care compositions herein may contain an emulsifier comprising one or more biobased alkyl ether sulfates of the general formula:

R₁—(OCH₂CH₂)x—O—SO₃M

Where:

R₁ is a non-petroleum derived, linear or branched C₈ to about C₂₀ (e.g. C₈ to about C₁₆, C₁₀ to C₁₄, or about C₁₂) alkyl moiety;

x is 0.5 to about 8 (e.g., 0.5 to 5, or 0.5 to 3); and

M is an alkali metal, ammonium, alkyl ammonium, or alkanol ammonium cation.

The fatty alcohol portion of the biobased alkyl ether sulfate (R₁) used herein are derived from a plant or animal source rather than geologically derived (i.e., petroleum based). For example, the fatty alcohol portion of the alkyl ether sulfate may be derived from coconut oil, palm kernel oil, palm oil, or a mixture of these. These oils contain relatively high concentrations of triglycerides and free fatty acids having chain lengths ranging from C₁₀ C₁₈, particularly C₁₀ to C₁₆, which may be especially desirable in the present compositions. The ethylene oxide portion of the biobased alkyl ether sulfate used herein may be biobased. Examples of biobased ethylene oxide include bioethanol-based ethylene oxide manufactured by Croda and bagasse-based ethylene oxide manufactured by Acme Hardesty. Other examples of biobased alkyl ether sulfates include CALFOAM from Pilot Chemical; EMAL LEVENOL, and LATEMAL from Kao Corporation; and POLYSTEP from Stepan. Other disclosure of biobased alkyl ether sulfates are described in U.S. Pat. No. 9,796,948. Alkyl ether sulfates may have a distribution of alkyl chain lengths (R₁) and/or degree of ethoxylation (x). In one embodiment, both the alkyl moiety (R₁) and the ethylene oxide portion of the biobased alkyl ether sulfate are biobased.

In some instances, the natural vaginal care compositions herein may contain an emulsifier comprising one or more fatty alcohol ethoxylates of the general formula:

R₂—(OCH₂CH₂)y—OH

Where:

R₂ is a non-petroleum derived, linear or branched C₁₀ to C₁₈, (e.g., C₁₂ to C₁₆ or C₁₂ to C₁₄) alkyl moiety); and

y is 0.5 to 250 (e.g., 2 to 200 or 5 to 150).

The fatty alcohol portion of the biobased fatty alcohol ethoxylate (R₂) used herein are derived from a plant or animal source, which may be the same or different plant and/or animal sources as used for the biobased alkyl ether sulfate (e.g., coconut oil, palm kernel oil, palm oil, or a mixture of these). The ethylene oxide portion of the biobased fatty alcohol ethoxylate used herein may be derived from biobased sources (e.g., bioethanol-based ethylene oxide manufactured by Croda and bagasse based ethylene oxide manufactured by Acme Hardesty). Fatty alcohol ethoxylates may have a distribution of alkyl chain lengths (R₂) and/or degree of ethoxylation (y). In some instances, both the alkyl moiety (R₂) and the ethylene oxide portion of the biobased fatty alcohol ethoxylate are biobased.

In some instances, the natural vaginal care compositions herein may contain a nonionic emulsifier comprising an alkyl polyglycoside of the general formula:

R3—O-Gz-

Where:

G is a moiety derived from reducing a saccharide containing 5 or 6 carbon atoms (e.g., glucose, fructose, mannose, galactose, talose, gulose, allose, altrose, idose, arabinose, xylose, lyxose or ribose).

R₃ is an alkyl group containing 6 to 20 carbon atoms (e.g., C₈ to C₁₆), which may be linear or branched and saturated or unsaturated; and

z is the degree of polymerization of the polyglycoside, representing the number of monosaccharide repeating units in the polyglycoside and has a value of less than 2.5 (e.g., between 1 and 2 or from 1.4 to 1.6).

The biobased alkyl polyglycosides for use herein are fatty ester derivatives of saccharides or polysaccharides formed when a carbohydrate is reacted under acidic condition with a fatty alcohol through condensation polymerization. Biobased alkyl polyglycosides may be derived from corn-based carbohydrates and fatty alcohols from natural oils in animals, coconuts and palm kernels. Alkyl polyglycosides may have a distribution of glucose units and/or alkyl chain lengths (R₃). Commercially available alkyl polyglycosides can be obtained as concentrated aqueous solutions ranging from 50 to 70% actives and are available from Cognis. Alkyl polyglycosides are also biodegradable in both anaerobic and aerobic conditions.

In some instances, the vaginal care composition includes nonionic biobased emulsifiers. The nonionic emulsifiers may stabilize oil-in-water emulsions and minimize irritation to sensitive vaginal tissue when applied by a female user. In one embodiment, the vaginal care composition comprises a biobased alkyl polyglycoside and a biobased ethoxylated alkyl polyglycoside emuslifier. A combination of emulsifiers with different hydrophilic headgroups may improve emulsion stability by enabling a wide range of oil water interfacial tensions.

### Biobased Preservatives

The presence of water can increase the susceptibility of a product to bacterial and/or fungal growth, which can reduce the shelf-life of the effected product. Thus, it may be desirable to include a biobased preservative to help inhibit the growth of undesirable microorganisms (e.g., bacteria and fungi). The compositions herein may include 0.05% to 10% (e.g., 0.1% to 5% or even 0.2% to 2%) of one or more biobased preservatives.

In some instances, the vaginal care composition includes a ferment-based preservative. Ferment-based preservatives can be obtained from the byproducts of microbial fermentation. Some nonlimiting examples of ferment-based preservatives include polylysine (e.g., epsilon-polylysine available from DKSH North America, Inc. and Epsiliseen® available from Siveele); Nisin™ available from Foodchem International Co.; and lactobacillus ferment (e.g., Leucidal® Liquid SF from Active Micro Technologies LLC).

In some instances, the vaginal care composition includes an extract-based preservative. Extract-based preservatives can be obtained from plant or animal material using conventional solvent extraction techniques. Some nonlimiting examples of extract-based preservatives include acerola fruit extract (e.g., Actiphyte® of Acerola from Active Organics), Arctium lappa root extract (e.g., burdock root in butylene glycol—3222NBG from Bio-Botanica, Inc.); black walnut leaf extract (e.g., Actiphyte® of black walnut leaves from Active Organics); Calendula officinalis flower extract (e.g., Calendula flower in butylene glycol—3224NBG from Bio-Botanica, Inc.), Camellia sinensis leaf extract (Green Tea CG from Sabinsa Cosmetics); grapefruit seed extract (e.g., Actiphyte™ grapefruit seed from Lipotec S.A.U.); hops extract (e.g., Viapure® Hops from Gnf-Selco); Japanese honeysuckle extract; honey (e.g., manuka honey), oregano extract (Microcurb™ OC from Kemin); rosemary extract (e.g., Herbalox® from Kalsec); Wasabi japonica root extract (e.g., Creanatural® from The Innovation Company); curry oil and cinnamon oil (e.g., Salinaturals® CCL from Ampak Co. Inc.); orange oil, lemon grass oil, and sesame oil (e.g., Salinaturals® OLG from Ampak Co. Inc.); basil extract, clove oil, lemon grass oil and sesame oil (e.g., Salinaturals® BCLS from Ampak Co. Inc.); turmeric oil, oregano extract, thyme extract, cinnamon bark, olive extract, rosemary extract, and peppermint extract (e.g., Suprapein® 8570 from Bio-Botanica, Inc.); willow bark extract (e.g., ABS willow bark extract from Active Concepts), Naticide® brand plant-derived perfume commercially available from Sinerga; and glucose oxidase and lactoperoxidase enzymes purified from milk (e.g., Biovert™ from Lonaz).

### pH Adjusters

It may be desirable to adjust the pH of the composition to a desired level or maintain a desired pH, for example, during product shipping or storage. The vaginal care compositions herein may have a pH of 3.5 to 7 (e.g., 3.8 to 6, 4.1 to 5.5, or 4.3 to 5). Without wishing to be bound by theory, it is believed that a vaginal care composition that is pH adjusted to a level similar to that of vaginal tissue may reduce irritation. This may further be improved by buffering the vaginal care composition pH using both the acid and a salt form of the same organic acid (e.g., citric acid and sodium citrate). A vaginal care composition buffered to a pH from about 3.5 to about 6 that also includes a biobased preservative may better resist microbial and/or fungal growth and be less irritating to sensitive vaginal tissue.

In some instances, a biobased acid may be used to modify the pH of the present compositions. Biobased acids may be extracted from plants, synthesized using microbial fermentation, or synthesized from plant extracts or microbial fermentation products. Examples of biobased acids include citric acid, lactic acid, succinic acid, 3-hydroxypropionic acid, glycolic acid, and glucaric acid. In some instances, the salt of a biobased acid (e.g., sodium citrate) may also be used to buffer the pH of the vaginal care composition. The compositions herein may include 0.01% to 5% of a pH adjuster.

### Other Optional Ingredients

The natural vaginal care compositions herein may include one or more vitamins and/or pro-vitamins (i.e., a substance that is converted into a vitamin within an organism) for providing a vaginal skin health benefit. Some non-limiting examples of vitamins and pro-vitamins that may be suitable for use herein are disclosed in U.S. Pat. No. 9,676,696. Some particularly suitable examples of vitamins and pro-vitamins that may be suitable for use herein include vitamin B 1, panthenol, vitamin B3, vitamin B5, vitamin E, and derivatives thereof (e.g., tocopheryl acetate). Vitamin(s) and/or pro-vitamin(s) may be included in the vaginal care composition at an amount of 0.1% to 7% (e.g., 0.5% to 5%, or even 2% to 4%) by weight of the vaginal care composition.

The vaginal care composition may comprise one or more humectants to provide a moisturizing benefit. An exemplary class of humectants is polyhydric alcohols. Suitable polyhydric alcohols include polyalkylene glycols and alkylene polyols and their derivatives, including propylene glycol, dipropylene glycol, polypropylene glycol, polyethylene glycol; sorbitol; hydroxypropyl sorbitol; erythritol; threitol; pentaerythritol; xylitol; glucitol; mannitol; hexylene glycol; butylene glycol (e.g., 1,3-butylene glycol); pentylene glycol; hexane triol (e.g., 1,2,6-hexanetriol); glycerin; ethoxylated glycerin; and propoxylated glycerin. The humectant may be present at 1% to 20% (e.g., 5% to 15%, or 8% to about 12%) by weight of the vaginal care composition.

The vaginal care composition may include 0.01% to 5% (e.g., 0.1% to 5%, 0.25% to 4%, 0.5% to 3%, or even 0.75% to 2%) by weight of a material for providing a desirable feel property to the composition. For example, the composition may include spherical or non-spherical particles that have a mean particle size of less than 125 μm (e.g., less than 100 μm, 75 μm, 50 μm, 40 μm, 30 μm, 20 μm, or even less than 15 μm). In some instances, it may be desirable to select a particle size of between 2 μm and 40 μm (e.g., 10 to 25 μm). If the particles are too big, they may feel abrasive when the composition is applied to sensitive vaginal skin. Particle size can be determined by any suitable method known in the art, such as by using Coulter counter equipment, laser diffraction equipment (e.g., LA-960 from Horiba Scientific or the like), dynamic or static image analysis equipment (e.g., Camsizer® from Horiba Scientific or the like), or optical microscopy (e.g., ASTM designation E20-85 titled “Standard Practice for Particle Size Analysis of Particulate Substances in the Range of 0.2 to 75 Micrometers by Optical Microscopy” ASTM Volume 14.02, 1993).

Some non-limiting examples of particle material that may be suitable for use herein include spherical particles of polyamide (e.g., nylon-12 and Orgasol™ 2002D Nat C05 from Atochem); ethylene acrylate copolymer (e.g., FloBead™ EA209 from Kobo); aluminum starch octenylsuccinate (e.g., the Dry Flo™ series of coated and uncoated spherical starch particles from Akzo Nobel); microspheres of polyethylene (e.g., Microthene™ FN510-00 from Equistar), and combinations of these. Still other examples of materials that may provide desirable feel properties include biobased, low-melting-point waxes. The low-melting-point wax may be selected to impart a dry feel when touched initially and a lubricious feel in use (i.e., upon melting). The low-melting-point wax, upon melting, may also facilitate the ease of spreading, rubbing or otherwise applying the vaginal care composition to vaginal tissue. The melting point of the low-melting-point waxes herein is selected to be less than the average human body temperature, which is typically about 37° C. For example, the low-melting-point wax may have a melting point of between 25° C. and 37° C. (e.g., 28° C. to 35° C.), which can be determined using the drop melting point described in ASTM D127.

### Methods of Use

The natural vaginal care compositions herein can be used to treat, for example, the symptoms of vaginal atrophy by applying the vaginal care composition to the vaginal tissue (e.g., labia and introitus) of a user who is experiencing symptoms of vaginal atrophy (e.g., a peri-menopausal, menopausal, or post-menopausal user) or vaginal dryness. The amount of the vaginal care composition applied to the vaginal tissue may vary, depending on, for example, the amount and/or type of vaginal care agent present in the composition and/or the symptom being treated. In some instances, a user may apply between 0.1 g and 2 g (e.g., 0.2 g to 1.2 g, or about 1 g) to the target vaginal tissue over the course of a treatment period. The vaginal care composition may be applied once a day, twice a day, or on a more frequent daily basis, during a treatment period. The treatment period is ideally of sufficient time for the vaginal care agent to provide the desired benefit. For example, the treatment period may last for at least 1 week (e.g., about 2 weeks, 4 weeks, 8 weeks, or even 12 weeks). In some instances, the treatment period will extend over multiple months (i.e., 3-12 months) or multiple years. In some instances, the vaginal care composition may be applied most days of the week (e.g., at least 4, 5 or 6 days a week), at least once a day or even twice a day during a treatment period of at least 2 weeks, 4 weeks, 8 weeks, or 12 weeks.

The vaginal care compositions herein may be applied by any suitable means known for applying such products, including digitally (i.e., with the hands and/or fingers) and/or with a disposable or reusable implement. The user may dispense the vaginal care composition onto a finger, hand, and/or implement and then apply the vaginal care composition to the target vaginal tissue. Additionally or alternatively, the user may dispense the vaginal care composition directly onto the target vaginal tissue and use a finger, hand, and/or implement to spread the vaginal care composition. Some non-limiting example of implements for applying a vaginal care composition include gloves, swabs, wipes, sponges, applicators with a cone-shaped insertion portion, plunger-style applicators, and vaginal rings. A particularly suitable example of an applicator with a cone-shaped insertion portion is the egg-shaped applicator described in co-pending U.S. Provisional Ser. No. 62/622,298. Exemplary methods of using an implement to apply a vaginal care composition are also disclosed in U.S. Provisional Ser. No. 62/622,298.

### Example Formulations

Table 1 provides examples of the vaginal care compositions described herein. The composition in Table 1 are oil-in-water emulsions. One and a half kilogram batches of Examples 1-12 are made gravimetrically according to the following procedure:

Aqueous phase (A) is prepared by combining all ingredients in a beaker and heating to 75° C. while stirring with a suitable mixer. Oil Phase (B) is prepared in a separate glass beaker by combining all ingredients and heating to 75° C. while stirring with a suitable stir plate and magnetic stir bar. Acidic aqueous phase (C) is prepared in separate glass beaker by combining all ingredients and heating to 40° C. while stirring with a suitable stir plate and magnetic stir bar.

Once Oil Phase (B) reaches 75° C., it is poured into the Water Phase (A) and milled with a Tekmar mixer for 5 minutes at 10,000 rpm, creating an oil-in-water emulsion. After milling, the acidic aqueous phase (C) is added, the heat is turned off, and the mixture is allowed to cool while stirring with an overhead mixer. When the mixture reaches 60° C., the remaining Finishing Ingredients (D) are added and the mixture is milled with a Tekmar mixer for 2 minutes at 8000 rpm. The resulting oil-in-water emulsion is transferred to a storage container and cooled to room temperature.

TABLE 1

| | Ex 1 | Ex 2 | Ex 3 | Ex 4 | Ex 5 | Ex 6 |
|---|---|---|---|---|---|---|
| Ingredient | wt. % | wt. % | wt. % | wt. % | wt. % | wt. % |
| Aqueous Phase (A) | | | | | | |
| Distilled Water | 67.55 | 62.02 | 60.07 | 64.90 | 40.00 | 47.70 |
| Glycerin | 7.00 | 12.00 | 5.00 | 6.00 | 2.00 | 10.00 |
| Sodium Hyaluronate¹ | 0.10 | 1.00 | 2.00 | 0.75 | 0.50 | 1.00 |
| Disodium EDTA² | 0.05 | 0.20 | 0.00 | 0.00 | 0.10 | 0.10 |
| Sodium Benzoate | 0.00 | 0.18 | 0.00 | 0.00 | 0.00 | 0.10 |
| Sodium salicylate | 0.20 | 0.00 | 0.00 | 0.00 | 0.25 | 0.05 |
| Bamboo | 0.50 | 0.00 | 0.00 | 0.00 | 0.00 | 0.00 |
| Cucumber (Cucumis sativus) | 0.00 | 0.00 | 3.00 | 0.00 | 0.00 | 0.00 |
| Sugar Cane | 0.50 | 1.00 | 0.00 | 0.00 | 0.00 | 0.00 |
| Xanthan gum | 1.50 | 0.00 | 0.00 | 2.00 | 0.00 | 1.00 |
| Cassava Strach | 0.00 | 2.00 | 0.00 | 0.00 | 0.00 | 0.00 |
| Tapioca Starch | 0.00 | 0.00 | 2.50 | 0.00 | 0.00 | 0.00 |
| Oil Phase (B) | | | | | | |
| Apricot Kernal Oil | 1.50 | 0.00 | 0.00 | 0.00 | 1.00 | 0.00 |
| Chamomile Oil | 0.00 | 1.00 | 0.00 | 0.00 | 0.00 | 0.00 |
| Eucalyptus Oil | 0.00 | 0.50 | 0.00 | 0.00 | 0.00 | 0.00 |
| Coconut Oil | 2.00 | 0.00 | 0.00 | 0.00 | 0.00 | 0.00 |
| Grape Seed Oil | 0.00 | 0.00 | 0.00 | 0.00 | 0.00 | 2.00 |
| Sweet Almond Oil | 0.00 | 0.00 | 2.00 | 0.00 | 0.00 | 0.00 |
| Peach Kernal Oil | 0.00 | 0.00 | 2.00 | 0.00 | 0.00 | 0.00 |
| Ylang Ylang Oil | 0.00 | 0.00 | 0.00 | 2.50 | 0.00 | 0.00 |
| Avocado Oil | 0.00 | 0.00 | 0.00 | 3.00 | 0.00 | 0.00 |
| Shea Butter | 0.00 | 0.00 | 0.00 | 0.00 | 1.00 | 0.00 |
| Olive | 0.00 | 0.00 | 0.00 | 2.00 | 0.00 | 0.00 |
| Aloe | 0.00 | 0.00 | 0.00 | 0.00 | 5.00 | 0.00 |
| Evening Primrose | 0.00 | 0.00 | 0.00 | 0.00 | 0.00 | 2.00 |
| Rose Hip | 0.00 | 0.00 | 0.00 | 0.00 | 0.00 | 1.00 |
| Cetearyl Glucoside, Cetearyl Alcohol³ | 0.25 | 0.20 | 0.20 | 0.75 | 0.20 | 0.25 |
| Cetyl Alcohol | 0.50 | 0.70 | 0.60 | 0.70 | 0.60 | 0.50 |
| Stearyl Alcohol | 1.20 | 1.00 | 0.90 | 1.10 | 0.90 | 1.20 |
| Behenyl Alcohol | 0.80 | 0.80 | 0.90 | 0.50 | 1.20 | 0.50 |
| Peg-100 Stearate⁴ | 0.10 | 0.10 | 0.10 | 0.05 | 0.15 | 0.20 |
| Castanea Sativa | 0.00 | 0.00 | 0.00 | 0.00 | 4.00 | 0.00 |
| Acidic Aqueous Phase (C) | | | | | | |
| Distilled Water | 10.00 | 11.00 | 14.00 | 9.00 | 15.00 | 10.00 |
| Citric Acid | 0.50 | 0.50 | 0.70 | 0.70 | 0.50 | 0.50 |
| Sodium Citrate | 0.60 | 0.60 | 0.85 | 0.85 | 0.60 | 0.60 |
| Finishing Phase (D) | | | | | | |
| Distilled Water | 5.00 | 5.00 | 5.00 | 5.00 | 25.00 | 20.00 |
| Phenoxyethanol | 0.15 | 0.00 | 0.00 | 0.00 | 0.00 | 0.10 |
| Sorbitan Caprylate⁵ | 0.00 | 0.20 | 0.18 | 0.20 | 0.00 | 0.20 |
| Gum acacia | 0.00 | 0.00 | 0.00 | 0.00 | 0.00 | 1.00 |
| Iota-Carrageenan | 0.00 | 0.00 | 0.00 | 0.00 | 2.00 | 0.00 |

| | Ex. 7 | Ex. 8 | Ex. 9 | Ex. 10 | Ex. 11 | Ex. 12 |
|---|---|---|---|---|---|---|
| Ingredient | wt. % | wt. % | wt. % | wt. % | wt. % | wt. % |
| Aqueous Phase (A) | | | | | | |
| Distilled Water | 71.25 | 62.10 | 55.90 | 69.90 | 39.05 | 42.95 |
| Glycerin | 7.00 | 12.00 | 5.00 | 6.00 | 2.00 | 10.00 |
| Sodium Hyaluronate¹ | 0.10 | 1.00 | 2.00 | 0.75 | 0.50 | 1.00 |
| Sodium salicylate | 0.00 | 0.00 | 0.00 | 0.00 | 0.10 | 0.05 |
| Bamboo | 0.50 | 0.00 | 0.00 | 0.00 | 0.00 | 0.00 |
| Cucumber | 0.00 | 1.00 | 3.00 | 0.00 | 0.00 | 0.00 |
| Sugar Cane | 1.00 | 0.00 | 0.50 | 0.00 | 0.00 | 0.00 |
| Xanthan gum | 1.00 | 0.00 | 0.00 | 2.00 | 0.00 | 1.00 |
| Cassava Strach | 0.50 | 2.00 | 0.00 | 0.00 | 0.00 | 0.00 |
| Tapioca Starch | 0.00 | 0.00 | 2.50 | 0.00 | 0.00 | 0.00 |
| Oil Phase (B) | | | | | | |
| Apricot Kernal Oil | 0.00 | 0.00 | 1.00 | 0.00 | 0.00 | 0.00 |
| Chamomile Oil | 2.00 | 1.00 | 0.00 | 0.00 | 0.50 | 0.00 |
| Eucalyptus Oil | 0.00 | 0.00 | 1.50 | 0.00 | 0.00 | 0.00 |
| Coconut Oil | 2.00 | 0.00 | 0.00 | 0.00 | 0.00 | 2.00 |
| Grape Seed Oil | 0.00 | 1.00 | 0.00 | 0.00 | 3.00 | 1.00 |
| Sweet Almond Oil | 0.00 | 0.00 | 2.00 | 0.00 | 0.00 | 0.00 |
| Peach Kernal Oil | 0.00 | 0.00 | 2.00 | 0.00 | 0.00 | 0.00 |
| Ylang Ylang Oil | 0.00 | 0.00 | 0.00 | 2.50 | 0.00 | 0.00 |
| Avocado Oil | 0.00 | 1.00 | 0.00 | 3.00 | 0.00 | 0.00 |
| Shea Butter | 0.00 | 0.00 | 0.00 | 0.00 | 1.00 | 1.00 |
| Olive | 0.00 | 0.00 | 1.50 | 2.00 | 0.00 | 0.00 |
| Aloe | 0.00 | 0.00 | 0.00 | 0.00 | 5.00 | 0.00 |
| Evening Primrose | 0.00 | 0.50 | 0.00 | 0.00 | 0.00 | 2.00 |
| Rose Hip | 0.00 | 0.00 | 0.00 | 0.20 | 0.00 | 1.00 |
| Cetearyl Glucoside, Cetearyl Alcohol³ | 0.25 | 0.20 | 0.50 | 1.00 | 0.20 | 0.25 |
| Cetyl Alcohol | 0.50 | 0.70 | 0.60 | 0.70 | 0.60 | 0.50 |
| Stearyl Alcohol | 1.20 | 1.00 | 0.90 | 1.10 | 0.90 | 1.20 |
| Behenyl Alcohol | 0.80 | 0.80 | 0.90 | 0.50 | 1.20 | 0.50 |
| Peg-100 Stearate⁴ | 0.20 | 0.10 | 0.10 | 0.05 | 0.15 | 0.20 |
| Castanea Sativa (Sweet Chestnut) | 0.00 | 0.00 | 0.00 | 0.00 | 4.00 | 0.00 |
| Acidic Aqueous Phase (C) | | | | | | |
| Distilled Water | 10.00 | 11.00 | 14.00 | 9.00 | 10.00 | 10.00 |
| Citric Acid | 0.50 | 0.50 | 0.50 | 0.50 | 0.50 | 0.50 |
| Sodium Citrate | 0.60 | 0.60 | 0.60 | 0.60 | 0.60 | 0.60 |
| Finishing Phase (D) | | | | | | |
| Distilled Water | 0.00 | 0.00 | 0.00 | 0.00 | 25.00 | 20.00 |
| Gum acacia | 0.00 | 0.00 | 0.00 | 0.00 | 2.00 | 1.00 |
| Iota-Carrageenan | 0.00 | 0.00 | 0.00 | 0.00 | 1.00 | 0.00 |
| Grapefruit Seed Extract⁶ | 0.60 | 0.00 | 0.00 | 0.00 | 0.20 | 0.00 |
| Willow Bark Extract⁷ | 0.00 | 3.00 | 0.00 | 0.00 | 2.50 | 0.00 |
| Japanese Honeysuckle Extract | 0.00 | 0.00 | 5.00 | 0.00 | 0.00 | 3.00 |
| Rosemary Extract⁸ | 0.00 | 0.00 | 0.00 | 0.20 | 0.00 | 0.25 |

¹Available as Bio-Sodium Hyaluronate Powder from SK Bioland (South Korea)
²Available as Dissolvine ® Na2-S from Akzo Nobel Functional Chemicals (Chicago, IL)
³Available as Emulgade ® PL68/50 from BASF (Cincinnati, OH)
⁴Available as Lipopeg ® 100-S from Lipo Chemicals (Warren Township, NJ)
⁵Available as Velsan ® SC from Clariant (Muttenz, Switzerland)
⁶Available as Actiphyte ™ grapefruit seed from Lipotec S.A.U.
⁷Available as ABS willow bark extract from Active Concepts
⁸Available as HERBALOX ® rosemary extracts from Kalsec

### Examples and Combinations

A. A natural vaginal care composition, comprising:
   a) a biobased vaginal care agent;
   b) a biobased thickener; and
   c) at least 60% water.
B. The composition of paragraph A, wherein the vaginal care agent comprises at least one of a biobased vaginal care chronic active and a biobased emollient.
C. The composition of paragraph B, wherein the biobased vaginal care active is selected from Castanea sativa oil, bamboo, Oenothera biennis, Humulus lupulus, Cucumis sativus, Aloe perfoliate, Olea europaea, sugar cane, rose hip, and mixtures of these.
D. The composition of paragraph B or C, wherein the biobased vaginal care active is present at about 0.0001% to about 15% by weight of the vaginal care composition.
E. The composition of paragraph B, wherein the biobased emollient is a botanical oil.
F. The composition of paragraph E, wherein the botanical oil is selected from calendula oil, chamomile oil, eucalyptus oil, rose hips oil, tea tree oil, ylang ylang oil, apricot kernel oil, avocado oil, cocoa butter, coconut oil, grape seed oil, moringa oil, olive oil, peach kernel oil, shea butter, sweet almond oil, sunflower oil, safflower oil, evening primrose oil, coconut oil, and mixtures of these.
G. The composition of paragraph E or F, wherein the emollient has an oil stability index of at least about 10 hours at 120° C.
H. The composition of any one of paragraphs E to G, wherein the emollient is selected from canola oil having an oleic content greater than 70, manila kernel oil, palm oil, palm olein, palm stearin, palm superolein, pecan oil, pumpkin seed oil, safflower oil having an oleic content of greater than about 30% and omega-6 fatty acid content of less than 50%, sesame oil, oleic soybean oil, sunflower oil having an oleic content of greater than about 40%, and mixtures thereof.
I. The composition of paragraph B or any one of paragraphs D to H, wherein the biobased emollient is present at about 0.1% to about 30% by weight of the composition.
J. The composition of any preceding paragraph, wherein the composition is an oil-in-water emulsion.
K. The composition of paragraph J, further comprising a biobased emulsifier.
L. The composition of any preceding paragraph, wherein the composition has a percent biobased carbon content of at least 80.
M. The composition of claim 1, wherein the biobased thickener is selected form biobased waxes, starches, gums, celluloses, and mixtures thereof.
N. The composition of any preceding paragraph, wherein the vaginal care composition is substantially free of vaginal irritants selected from retinoids, ethanol, sunscreen agents, perfumes, estrogen, progesterone, particulates having an average particle size of greater than about 125 microns, and combinations of these.
O. The composition of any preceding paragraph, further comprising a biobased wax that has a melting point of between about 25° C. and about 37° C.
P. The composition of any preceding paragraph, further comprising about 0.05% to about 10% of a biobased preservative.
Q. The composition of paragraph P, wherein the biobased preservative includes at least one of a ferment-based preservative and an extract-based preservative.
R. The composition of any preceding paragraph, further comprising about 0.01% to about 5% of a biobased pH adjuster.
S. The composition of paragraph R, wherein the biobased pH adjuster includes at least one of a biobased acid and a salt of a biobased acid.
T. A method of treating a vaginal condition comprising:
   a) identifying a target portion of vaginal tissue exhibiting a sign of a vaginal condition in need of treatment;
   b) applying an effective amount of the composition of claim 1 to the target portion of vaginal tissue during a treatment period, wherein the treatment period is sufficient for the composition to improve the vaginal condition.
U. The method of paragraph T, wherein the vaginal condition is vaginal atrophy or vaginal dryness, and the vaginal care composition is applied to at least one of the vaginal introitus and the labia majora.
V. The method of paragraph U, wherein the vaginal care composition comprises an effective amount of a vaginal care agent selected from lecithin, Humulus lupulus, Alpinia speciose, Plantago lanceolate, calcitriol, diisopropyl adipate, kubi extract, activated Micrococcus luteus, mitoquinol mesylate, Castanea sativa, hydrolyzed rice, myristoyl hexapeptide-16, dextrin, tetrapeptide-26, Sambucus nigra, and combinations thereof.
W. The method of any one of paragraphs T to V, wherein the female user is menopausal or post-menopausal.
X. The method of any one of paragraphs T to W, wherein the vaginal care composition is applied with a topical applicator selected from gloves, swabs, wipes, sponges, plunger-style applicators, egg-shaped applicators, and vaginal rings.
Y. A natural vaginal care composition, comprising:
   a) 0.01% to 40% of a biobased vaginal care agent;
   b) 0.01% to 10% of a biobased thickener;
   c) 0.01% to 10% of a biobased emulsifier; and
   d) at least 60% water, wherein the natural vaginal care composition is an oil-in-water emulsion.
Z. The natural vaginal care composition of paragraph Y, wherein the vaginal care agent comprises a biobased emollient.
AA. The natural vaginal care composition of paragraph Y or Z, wherein the vaginal care agent comprises a botanical oil.
BB. The natural vaginal care composition of any one of paragraphs T to AA, wherein the vaginal care agent comprises a biobased chronic active.
CC. A natural vaginal care composition, comprising:
   a) about 0.5 to about 30% by weight of a biobased emollient;
   b) about 0.01 to about 10% by weight of an emulsifier selected from biobased fatty alcohol ethoxylates, biobased alkyl polyglycosides, and mixtures thereof; and
   c) at least 60% water, wherein the composition has a pH of about 3.5 to about 6.
DD. The composition of paragraph CC, wherein the biobased fatty alcohol ethoxylates have the structure: R₂—(OCH₂CH₂)y—OH, wherein
   a) R₂ is a linear or branched C₁₀ C₁₈ alkyl, and
   b) y is 0.5 to 250.
EE. The composition of paragraph DD, wherein at least one of R₂ and —(OCH₂CH₂)y—OH are biobased.
FF. The composition of paragraph CC, wherein the biobased alkyl polyglycosides have the structure: R₃—O-Gz, wherein
   a) G is derived from reducing at least one of glucose, fructose, mannose, galactose, talose, gulose, allose, altrose, idose, arabinose, xylose, lyxose and ribose,
   b) R₃ is a biobased linear or branched C₆ to C₂₀ alkyl, and
   c) z is less than 2.5.
GG. A natural vaginal care composition, comprising:
   a) about 0.01% to about 40% of a biobased vaginal care agent
   b) about 0.1 to about 10% of an extract-based preservative selected from acerola fruit extract, Arctium lappa root extract, black walnut extract, Calendula officinalis flower extract, Camellia sinensis extract, grapefruit seed extract, hops extract, Japanese honeysuckle extract, manuka honey, oregano extract, rosemary extract, Wasabi japonica root extract, curry oil, cinnamon oil, orange oil, lemon grass oil, sesame oil, basil extract, clove oil, turmeric oil, thyme extract, cinnamon bark, olive extract, rosemary extract, peppermint extract, willow bark extract, and mixtures thereof;
   c) about 0.01 to about 5% of an acid selected from citric acid, lactic acid, succinic acid, 3-hydroxypropionic acid, glycolic acid, glucaric acid, salts of these, and mixtures thereof;
   d) at least 60% water; wherein the pH of the composition is about 3.5 to about 6.
HH. The composition of paragraph GG, wherein the acid is biobased.
II. The composition of paragraph GG or HH, wherein the composition comprises at least one of the acids and a salt of the acid.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

Whereas particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A natural vaginal care composition, comprising:
a) a biobased vaginal care agent;
b) a biobased thickener; and
c) at least 60% water.

2. The composition of claim 1, wherein the vaginal care agent comprises at least one of a biobased vaginal care chronic active and a biobased emollient.

3. The composition of claim 2, wherein the biobased vaginal care active is selected from Castanea sativa oil, bamboo, Oenothera biennis, Humulus lupulus, Cucumis sativus, Aloe perfoliate, Olea europaea, sugar cane, rose hip, and mixtures of these.

4. The composition of claim 3, wherein the biobased vaginal care active is present at about 0.0001% to about 15% by weight of the vaginal care composition.

5. The composition of claim 2, wherein the biobased emollient is a botanical oil.

6. The composition of claim 5, wherein the botanical oil is selected from calendula oil, chamomile oil, eucalyptus oil, rose hips oil, tea tree oil, ylang ylang oil, apricot kernel oil, avocado oil, cocoa butter, coconut oil, grape seed oil, moringa oil, olive oil, peach kernel oil, shea butter, sweet almond oil, sunflower oil, safflower oil, evening primrose oil, coconut oil, and mixtures of these.

7. The composition of claim 5, wherein the emollient has an oil stability index of at least about 10 hours at 120° C.

8. The composition of claim 7, wherein the emollient is selected from canola oil having an oleic content greater than 70, manila kernel oil, palm oil, palm olein, palm stearin, palm superolein, pecan oil, pumpkin seed oil, safflower oil having an oleic content of greater than about 30% and omega-6 fatty acid content of less than 50%, sesame oil, oleic soybean oil, sunflower oil having an oleic content of greater than about 40%, and mixtures thereof.

9. The composition of claim 2, wherein the biobased emollient is present at about 0.1% to about 30% by weight of the composition.

10. The composition of claim 1, wherein the composition is an oil-in-water emulsion.

11. The composition of claim 10, further comprising a biobased emulsifier.

12. The composition of claim 1, wherein the composition has a percent biobased carbon content of at least 80.

13. The composition of claim 1, wherein the biobased thickener is selected form biobased waxes, starches, gums, celluloses, and mixtures thereof.

14. The composition of claim 1, wherein the vaginal care composition is substantially free of vaginal irritants selected from retinoids, ethanol, sunscreen agents, perfumes, estrogen, progesterone, particulates having an average particle size of greater than about 125 microns, and combinations of these.

15. The composition of claim 1, further comprising a biobased wax that has a melting point of between about 25° C. and about 37° C.

16. The composition of claim 1, further comprising about 0.05% to about 10% of a biobased preservative.

17. The composition of claim 16, wherein the biobased preservative includes at least one of a ferment-based preservative and an extract-based preservative.

18. The composition of claim 1, further comprising about 0.01% to about 5% of a biobased pH adjuster.

19. The composition of claim 18, wherein the biobased pH adjuster includes at least one of a biobased acid and a salt of a biobased acid.

20. A method of treating a vaginal condition comprising:
a) identifying a target portion of vaginal tissue exhibiting a sign of a vaginal condition in need of treatment; and
b) applying an effective amount of the composition of claim 1 to the target portion of vaginal tissue during a treatment period, wherein the treatment period is sufficient for the composition to improve the vaginal condition.
